(19) United States
(12) Patent Application Publication
(10) Pub. No.: US 2025/0353832 A1
(43) Pub. Date: Nov. 20, 2025

(54) PROCESS FOR THE PREPARATION OF AMINOPYRIDAZINE DERIVATIVES

(71) Applicant: ADAMA MAKHTESHIM LTD., Beer Sheva (IL)

(72) Inventors: Eran FOGLER, Meitar (IL); Michael GRABARNICK, Meitar (IL); Revanappa Vasantrao GALGE, Udgir, Latur, Maharashtra (IN)

(73) Assignee: ADAMA MAKHTESHIM LTD., Beer Sheva (IL)

(21) Appl. No.: 19/119,542

(22) PCT Filed: Oct. 12, 2023
(86) PCT No.: PCT/IL2023/051070

§ 371 Date: Apr. 9, 2025

(30) Foreign Application Priority Data
Oct. 12, 2022 (IN) 202211058387

(51) Int. Cl. C07D 403/12; C07D 231/16; C07D 237/20

(52) U.S. Cl. CPC C07D 403/12; C07D 231/16; C07D 237/20

(57) ABSTRACT

The present invention provides a process for the preparation of aminopyridazine derivatives, and novel pyridazine-based and pyrazole-based compounds which are useful as either starting materials or intermediates in said process.

### TECHNICAL FIELD

The present invention provides a process for the preparation of aminopyridazine derivatives, as well as novel pyridazine-based and pyrazole-based compounds which are useful as either starting materials or intermediates in said process.

### BACKGROUND ART

Invertebrate pests, in particular arthropods and nematodes, destroy growing and harvested crops and attack wooden dwelling and commercial structures, thereby causing large economic loss to the food supply and to property.

Aminopyridazine derivatives have a good pesticidal activity against a broad spectrum of different invertebrate pests, especially difficult to control pests such as insects, and are therefore useful for combating invertebrate pests.

WO 2018/082964 discloses a process for the preparation of aminopyridazine derivatives by reacting pyridazine derivatives bearing an amino group at the 4-position of the pyridazine moiety with pyrazole derivatives bearing a carbonyl group at the 4-position of the pyrazole moiety, e.g., acyl halide- and carboxylic acid-pyrazole derivatives.

There is an ongoing need for developing new synthetic processes for preparing such compounds thereby providing alternative routes that enable to diverse the starting materials and intermediates used.

### SUMMARY OF INVENTION

The Experimental section herein shows the preparation of N-ethyl-5-methyl-1-(3-methylbutan-2-yl)-N-(pyridazin-4-yl)-1H-pyrazole-4-carboxamide. Specifically, in one flask, 4-bromo-5-methyl-1-(3-methylbutan-2-yl)-1H-pyrazole has been reacted with n-butyl lithium in the presence of a solvent at −78° C. (herein identified as “Reaction Mass A”); and in another flask, N-ethylpyridazin-4-amine has been reacted with a base such as trimethyl amine and triethyl amine in the presence of a solvent followed by a reaction with triphosgene (herein identified as “Reaction Mass B”). Then, Reaction Mass A was added to Reaction Mass B at −78° C. to obtain N-ethyl-5-methyl-1-(3-methylbutan-2-yl)-N-(pyridazin-4-yl)-1H-pyrazole-4-carboxamide.

In one aspect, the present invention thus relates to a process for the preparation of a compound of formula I

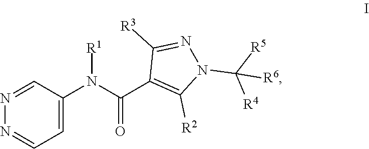

or a salt, N-oxide, tautomer, or enantiomer thereof,
wherein:
R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl;
R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl; and
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH,
said process comprising reacting a compound of formula III with either:
(i) a compound of formula II, to thereby obtain the compound of formula I; or
(ii) 4-isocyanatopyridazine, to thereby obtain the compound of formula I wherein R¹ is H, and optionally substituting said hydrogen with a group selected from (C₁-C₁₂)alkyl optionally interrupted with one or more groups each independently selected from —O—, —S—, and —N((C₁-C₁₂)alkyl)-, (C₂-C₆)alkenyl, and (C₃-C₇)cycloalkyl,

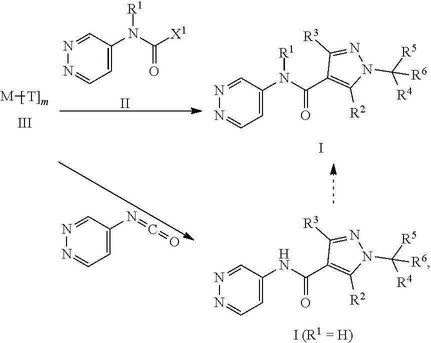

wherein:
T each independently is a group of the formula:

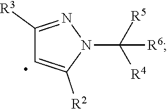

M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminum ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex, wherein M represents said metal ion connected to an additional metal ion (e.g., an alkali metal ion or alkali earth metal ion) optionally via either one or more halogen anions, or one or more of said T group(s);
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl);
n is an integer of 0-3;
m is an integer of 1-3; and
Cat⁺ is a cation such as an alkali metal cation.

In certain embodiments, disclosed herein is a process for the preparation of a compound of formula I, wherein M is a group of formula —Y—Z₍ₙ₎; and Y is a metal ion as defined above, having an oxidation state of 1, 2 or 3, i.e., the sum of n and m is 1, 2, or 3.

In another aspect, the present invention provides a compound of formula III

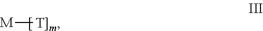

or a salt, N-oxide, tautomer, or enantiomer thereof,
wherein:
T each independently is a group of the formula:

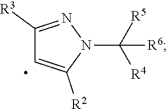

R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl;
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH;
M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminium ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex wherein M represents said metal ion connected to an additional metal ion (e.g., an alkali metal ion or alkali earth metal ion) optionally via either one or more halogen anions, or one or more of said T group(s);
n is an integer of 0-3;
m is an integer of 1-3; and
Cat⁺ is a cation such as an alkali metal cation, provided that when R² is methyl, R³ is H, R⁴ is —CH(CH₃)₂, R⁵ is methyl, and R⁶ is H, M is not —B(OH)₂; 5,5-dimethyl-1,3,2-dioxaborinan-2-yl; or 4,4,5,5-tetramethyl-1,3,2-dioxaborolane-2-yl.

In certain embodiments, disclosed herein is a compound of the formula III, wherein M is a group of formula —Y—Z₍ₙ₎; and Y is a metal ion as defined above, having an oxidation state of 1, 2 or 3, i.e., the sum of n and m is 1, 2, or 3.

In yet another aspect, the present invention provides a compound of formula II

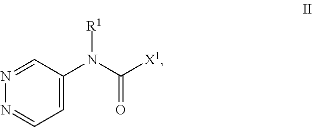

or a salt, N-oxide, or tautomer thereof,
wherein:
R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl; and
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl), provided that when R¹ is H, X¹ is not —O-phenyl.

In still another aspect, the present invention provides a compound of formula IV

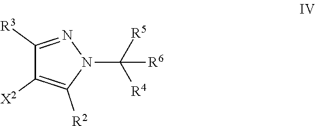

or a salt, N-oxide, or enantiomer thereof, wherein X² is halogen such as Cl and Br; and
(i) R² is methyl, R³ is H, R⁴ is —CH(CH₃)₂, R⁵ is methyl, R⁶ is H;
(ii) R² is methyl, R³ is H, R⁴ is —C(H)(F)CH₃, R⁵ is methyl, R⁶ is H;
(iii) R² is methyl, R³ is H, R⁴ is 1-cyanocyclopropyl, R⁵ is methyl, R⁶ is H; (iv) R² is methyl, R³ is H, R⁴ is methyl, R⁵ is methyl, R⁶ is H;
(v) R² is methyl, R³ is H, R⁴ is —CF₃, R⁵ is methyl, R⁶ is H;
(vi) R² is methyl, R³ is H, R⁴ is 1-carbamoylcyclopropyl, R⁵ is methyl, R⁶ is H; or
(vii) R² is methyl, R³ is H, R⁴ and R⁵ together represent 3,3-difluoro-1,5-pentylene, R⁶ is H.

In a further aspect, the present invention provides a composition comprising a compound of formula I as defined above, or a salt, N-oxide, tautomer, or enantiomer thereof, obtained by the process disclosed hereinabove, and at least one additional compound selected from:

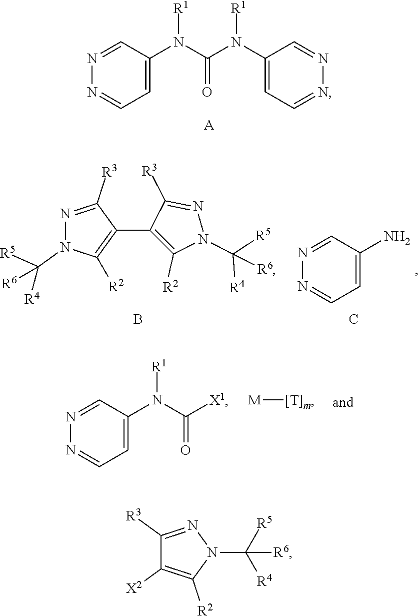

wherein:
T each independently is a group of the formula:

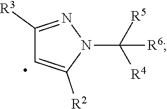

R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl;
R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl;
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH;
M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminium ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex wherein M represents said metal ion connected to an additional metal ion (e.g., an alkali metal ion or alkali earth metal ion) optionally via either one or more halogen anions, or one or more of said T group(s);
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl); X² is halogen such as Cl and Br;
n is an integer of 0-3;
m is an integer of 1-3; and
Cat⁺ is a cation such as an alkali metal cation.

In certain embodiments, disclosed herein is a composition as defined above, wherein said at least one additional compound comprises M [symbol] T]ₘ, wherein M is a group of formula —Y—Z₍ₙ₎; and Y is a metal ion as defined above, having an oxidation state of 1, 2 or 3, i.e., the sum of n and m is 1, 2, or 3.

### DETAILED DESCRIPTION

In one aspect, disclosed herein is a process for the preparation a compound of formula I as defined above, said process comprising reacting a compound of formula III as defined above with either: (i) a compound of formula II as defined above, to thereby obtain the compound of formula I; or (ii) 4-isocyanatopyridazine, to thereby obtain the compound of formula I wherein R¹ is H, and optionally substituting said hydrogen with a group selected from (C₁-C₁₂)alkyl optionally interrupted with one or more groups each independently selected from —O—, —S—, and —N((C₁-C₁₂)alkyl)-, (C₂-C₆)alkenyl, and (C₃-C₇)cycloalkyl.

The term “N-oxide”, as used herein referring to any one of the compounds of formulae I, II, III and IV, relates to a form of said compound wherein at least one, i.e., one or more, of the ring nitrogen atoms is oxidized (as NO). Such N-oxides may be prepared by standard methods, e.g., as described in Botteghi et al., Journal of Organometallic Chemistry, 1989, 370, 17-31.

The term “tautomer”, as used herein referring to any one of the compounds of formulae I, II and III, relates to a structural (constitutional) isomer of said compound that readily interconvert. Tautomers of the compound disclosed include, e.g., amide-imidic acid tautomers of said compound.

The term “enantiomer”, as used herein referring to any one of the compounds of formulae I, III and IV, refers to one of two stereoisomers that are mirror images of each other that are non-superposable (not identical).

The term “alkyl” typically means a linear or branched hydrocarbyl, i.e., a univalent group derived from a saturated linear or branched aliphatic chain by removal of hydrogen atom from any of the carbon atoms, having, e.g., 1-12 carbon atoms and includes methyl, ethyl, n-propyl, isopropyl, n-butyl, sec-butyl, isobutyl, tert-butyl, n-pentyl, 2,2-dimethylpropyl, n-hexyl, n-heptyl, n-octyl, n-nonyl, n-decyl, n-undecyl, n-dodecyl, and the like. The term “alkenyl” typically means a linear or branched hydrocarbyl having, e.g., 2-6 carbon atoms and one or more double bond, and includes ethenyl, propenyl, 3-buten-1-yl, 2-ethenylbutyl, 3-octen-1-yl, and the like. The term “haloalkyl” typically means an alkyl as defined herein, substituted with one or more groups each independently selected from a halogen.

The term “halogen” as used herein refers to a halogen and includes fluoro, chloro, bromo, and iodo, but it is preferably bromo or chloro.

The term “carbocyclic ring” as used herein refers to a mono-, bi-, or poly-cyclic non-aromatic hydrocarbon having, e.g., 3-12, but preferably 3-7, carbon atoms. The carbocyclic ring may be saturated, such as cyclopropane, cyclobutane, cyclopentane, cyclohexane, cycloheptane, and the like; or unsaturated, i.e., having at least one double bond, such as cyclopropene, cyclobutene, cyclopentene, cyclohexene, cycloheptene, and the like. The carbocyclic ring may be substituted, e.g., by one or more alkyl groups. The term “cycloalkyl” means a univalent group derived from a carbocyclic ring by removal of hydrogen atom from any of the carbon atoms. Examples of such groups include, without limiting, cyclopropyl, cyclobutyl, cyclopentyl, cyclohexyl, cycloheptyl, and the like.

The term “heterocyclic ring” denotes a mono- or poly-cyclic non-aromatic ring of, e.g., 3-7 atoms containing at least one carbon atom and one to three heteroatoms selected from oxygen, nitrogen and sulfur (optionally oxidized), which may be saturated or unsaturated, i.e., containing at least one unsaturated bond. Preferred are 5- or 6-membered heterocyclic rings. The term “heterocyclyl” as used herein refers to any univalent group derived from a heterocyclic ring as defined herein by removal of hydrogen from any ring atom. Examples of such groups include, without limitation, pyridinyl, pyrimidinyl, aziridinyl, piperidinyl, pyrrolidinyl, azepinyl, morpholinyl such as 4-morpholinyl, oxazolyl, dihydrooxazolyl, oxadiazolyl; imidazolyl, imidazolinyl, dihydroimidazolyl, pyrazolyl, triazolyl, tetrazolyl, thiadiazolyl, piperazinyl, tetrahydropirydinyl, and oxapinyl.

According to the process of the present invention, a compound of the formula III, consisting of a group M linked to 1, 2 or 3 T groups (M-[T]ₘ), is reacted with either (i) a compound of the formula II, to thereby obtain the compound of formula I; or (ii) 4-isocyanatopyridazine, to thereby obtain the compound of formula I wherein R¹ is H. In certain embodiments, group M is linked to a sole T group, i.e., m is 1. In other embodiments, group M is linked to more than one, i.e., 2 or 3, T groups, wherein said groups T may be either identical (wherein R², R³, R⁴, R⁵, and R⁶ are identical) or different.

In certain embodiments, M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminum ion, and a transition metal (e.g., copper, zinc, iron) ion, and Z represents a halogen anion, e.g., Cl⁻ or Br⁻.

In other embodiments, M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein Cat⁺ is a cation, e.g., an alkali metal cation; and R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl. Examples of such rings include, without being limited to, 5,5-dimethyl-1,3,2-dioxaborinan-2-yl, 4,4,5,5-tetramethyl-1,3,2-dioxaborolane-2-yl, benzo[d][1,3,2]dioxaborole-2-yl, 9-borabicyclo[3.3.1]non-9-yl, or 6-methyl-1,3,6,2-dioxazaborocane-4,8-dione-2-yl.

In further embodiments, M together with the T group(s) attached thereto form an ate complex, wherein M represents a metal ion having an oxidation state of at least one such as an alkali metal ion, an alkali earth metal ion, aluminum ion, and a transition metal ion, connected to an additional metal ion (e.g., an alkali metal ion or alkali earth metal ion) optionally via one or more halogen anions or one or more of said T group(s).

The term “ate complex” as used herein refers to a salt comprising a bimetallic system, wherein one of the metals has Lewis acidity higher than that of the second metal, for example wherein the former metal is Mg²⁺ and the latter is, e.g., Na⁺ or Li⁺, and the former metal is thus in interaction with the Lewis basic anionic ligands, i.e., said T group(s). Examples of ate complexes disclosed in the literature include the magnesate complex

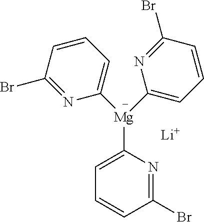

(Lida et al., Tetrahedron Letters, 2001, 42, 4841-4844), and the complex

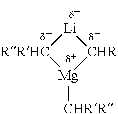

which is formed between dialkylmagnesium and alkyllithium (Carlotti et al., Polymer, 2009, 50, 3057-3067).

In certain embodiments, the process of the present invention is carried out at a temperature of from about −100° C. to about 70° C., e.g., from about −90° C. to about 50° C., from about −80° C. to about 30° C. or 40° C., or from about −78° C. to about 25° C.

In certain embodiments, the compound of formula III is reacted with a compound of formula II to thereby obtain the compound of the formula I.

In other embodiments, the compound of formula III is reacted with 4-isocyanatopyridazine to thereby obtain a compound of the formula I wherein R¹ is hydrogen. In some particular such embodiments, the compound obtained is reacted with a base, following by a reaction with an alkylation reagent, to thereby replace said hydrogen by (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl. In other particular such embodiments, the compound obtained following the reaction of the compound of formula III with 4-isocyanatopyridazine is reacted with said base and said alkylation reagent simultaneously.

The term “alkylation reagent” as used herein refers to an alkyl-bearing agent that is capable of effecting, e.g., reducing, the molecular polarity of a molecule having active hydrogen, by replacing said active hydrogen with an alkyl group. For instance, alkylation reagents could be used for forming a carbon-nitrogen bond, e.g., by alkylation of an amide to form N-alkyl amide. Non-limiting examples of alkylation reagents include ethyl iodide, methyl iodide, ethyl methanesulfonate, trimethyloxonium tetrafluoroborate, triethyloxonium tetrafluoroborate, triethyloxonium hexachloroantimonate, bromoethane, and diethylsulfate.

In certain embodiments, the compound of formula III is prepared from a compound of formula IV, or a mixture thereof, following the process:

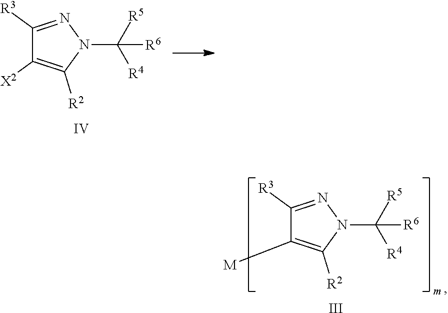

wherein:
X² is halogen such as Cl and Br; and
R², R³, R⁴, R⁵, R⁶, M, and m each independently is as defined in any one of the embodiments above, by substituting, i.e., replacing, the X² group in the compound of formula IV with the group -M-[T]m-i (also referred to herein as “a process for the preparation of the compound of formula III”). In certain particular such embodiments, the compound of the formula III is prepared from identical compounds of the formula IV, i.e., compounds of the formula IV having identical R², R³, R⁴, R⁵, R⁶, and X²groups, and the compound of the formula III thus obtained has the structure M-[T]ₘ, wherein m is an integer of 1-3; and the T groups are identical. In other particular such embodiments, the compound of the formula III is prepared from a mixture of different compounds of the formula IV, and the compound of the formula III thus obtained has the structure M-[T]ₘ, wherein m is an integer of 1-3; and the T groups are different.

According to the invention, the number of T groups bound to M, m, may be dependent on the solvent in which the compound of formula IV is reacted with the reagent comprising the group M. Suitable such solvents include, e.g., ethers such as diethyl ether, tetrahydrofuran (THF), 1,4-dioxane, methyl tert-butyl ether, and glycol dimethyl ether. Due to the prominent coordination of the oxygen atom in ethers to metals, ethers are extensively used in reactions involving organometallic reagents or intermediates such as the reaction to obtain the compound of the formula III from the compound of the formula IV.

In certain embodiments, the process for the preparation of the compound of formula III is carried out in THF, and the compound thus obtained mostly contains only one T group that is bound to M, i.e., m is 1. In other certain embodiments, said process is carried out in 1,4-dioxane, and the compound of formula (III) thus obtained mostly contains two T groups that are bound to M, i.e., m is 2.

In certain embodiments, the process to obtain the compound of formula III according to any one of the embodiments above is carried out at a temperature of from about −100° C. to about 100° C.

In certain embodiments, the compound of formula III is a Grignard reagent, i.e., a chemical compound of the formula T-Mg-Hal, wherein Hal is halogen, preferably Cl or Br; and T is as defined hereinabove, which is prepared by reacting the compound of formula IV, wherein X² is halogen, preferably Cl or Br, with solid magnesium or a magnesium-halogen exchange reagent such as i-PrMgCl and i-PrMgBr in a dry ether-based solvent such as diethylether or a mixture of dry ether-based solvent and dry non protic solvent such as toluene. Grignard reagents are popular reagents in organic synthesis for creating carbon-carbon bonds.

In other embodiments, the compound of formula III is in the form of an ate complex, more specifically an organocuprate reagent, e.g., Gilman reagent consisting of lithium, copper, and two T groups each independently as defined hereinabove, having the molecular formula T₂CuLi, which is prepared by reacting the compound of formula IV with lithium metal, following by reacting the compound obtained with copper halide, e.g., CuBr and CuI.

In further embodiments, the compound of formula III is an organolithium reagent of the formula T-Li, which is prepared by reacting the compound of formula IV with lithium metal or a lithium-based reagent of the formula Li—R, wherein R is an organic group such as alkyl. In particular such embodiments, M is a group of formula —Y—Z₍ₙ₎, wherein Y is lithium ion, and n is 0, and the process to obtain the compound of formula III according to any one of the embodiments above involves reacting the compound of formula IV with n-butyl lithium or n-hexyl lithium, in a solvent such as THF, at a temperature of from about −78° C. to about 0° C.

In certain embodiments, the compound of formula II is prepared from a compound of formula XI, following the process:

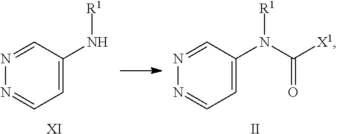

by substituting the nitrogen atom of the secondary amine group by a carbonyl-containing group of the formula —C(O)X¹, wherein R¹ and X¹ each independently is as defined hereinabove (also referred to herein as “a process for the preparation of the compound of formula II”). In particular embodiments, the compound of formula II is prepared by dissolving the compound of formula XI in a solvent, and then reacting said compound with a base at a temperature of from about −100° C. to about 50° C., e.g., from about −90° C. to about 40° C., from about −80° C. to about 30° C., or from about −78° C. to about 25° C., followed by a reaction with a carbonyl-containing reagent. Non-liming examples of suitable bases include trimethylamine, triethylamine, tributylamine, N,N-diisopropylethylamine, n-butyl lithium, t-butyl lithium, n-hexyl lithium, lithium diisopropylamide (LDA), potassium bis(trimethylsilyl)amide (KHMDS), sodium hydroxide (e.g., in the form of a solid or aqueous solution of at least 20% sodium hydroxide), potassium carbonate, sodium carbonate, and sodium hydride; and examples of carbonyl-containing reagents include, without limiting, phenyl chloroformate, phosgene, 1,1,1-trichloromethylformate (diphosgene), bis(trichloromethyl) carbonate (BTC; triphosgene), and phenyl 4,5-dichloro-6-oxopyridazine-1(6H)-carboxylate.

In certain embodiments, the process disclosed herein, according to any one of the embodiments above, is for the preparation of a compound of the formula I, wherein: (i) R¹ is ethyl, R² is methyl, R³ is H, R⁴ is —CH(CH₃)₂, R⁵ is methyl, and R⁶ is H; (ii) R¹ is ethyl, R² is methyl, R³ is H, R⁴ is —C(H)(F)CH₃, R⁵ is methyl, and R⁶ is H; (iii) R¹ is ethyl, R² is methyl, R³ is H, R⁴ is 1-cyanocyclopropyl, R⁵ is methyl, and R⁶ is H; (iv) R¹ is ethyl, R² is methyl, R³ is H, R⁴ is methyl, R⁵ is methyl, and R⁶ is H; (v) R¹ is ethyl, R² is methyl, R³ is H, R⁴ is —CF₃, R⁵ is methyl, and R⁶ is H; (vi) R¹ is ethyl, R² is methyl, R³ is H, R⁴ is 1-carbamoylcyclopropyl, R⁵ is methyl, and R⁶ is H; or (vii) R¹ is ethyl, R² is methyl, R³ is H, R⁴ and R⁵ together represent 3,3-difluoro-1,5-pentylene, and R⁶ is H.

In particular such embodiments, m is 1, i.e., only one T group is linked to group M. In certain more particular embodiments, M is Li ion, Cu ion, —Mg(Hal)₁, —Cu(Hal)₁, —Zn(Hal)₁, —BF₃⁻K⁺, —B(OH)₂, —B(—OiPr)₃Cat⁺, 5,5-dimethyl-1,3,2-dioxaborinan-2-yl, 4,4,5,5-tetramethyl-1,3,2-dioxaborolane-2-yl, benzo[d][1,3,2]dioxaborole-2-yl, 9-borabicyclo[3.3.1]non-9-yl, or 6-methyl-1,3,6,2-dioxazaborocane-4,8-dione-2-yl; and Hal is a halogen, preferably Cl or Br. In other more particular embodiments, M has the formula —Mg(Hal)₂Li, —Zn(Hal)₂Li, —Cu(Hal)₂Mg, or —Cu(Hal)₂Li, and together with the T group form an ate complex; Hal is a halogen, preferably Cl or Br; and the Mg, Zn or Cu ion is bound to the T group and connected to the Li or Mg ion via the halogen anions.

In other particular such embodiments, m is 2, i.e., two identical T groups are linked to group M. In more particular embodiments, M is Mg ion or Zn ion; or M has the formula CuLi, and together with the T groups form an ate complex wherein the Cu ion is bound to each one of the T groups.

In yet other particular such embodiments, m is 3, i.e., three identical T groups are linked to group M. In more particular such embodiments, M is boron ion, Al ion, or Fe ion; or M has the formula MgLi, and together with the T groups form an ate complex wherein the Mg ion is bound to each one of the T groups.

The process disclosed herein, according to any one of the embodiments above, may be carried out with different efficiencies, depending on the reagents and conditions used. In other words, the product of this process may comprise different percentages of the desired compound I, as well as certain amounts of other compounds which are either non-reacted starting material or side-product obtained by reactions other than the major one leading to the desired compound. In certain embodiments, the product obtained as a result of the process disclosed herein comprises, in addition to the compound of formula I, at least one of the by-products A, B and C (pyridazin-4-amine)

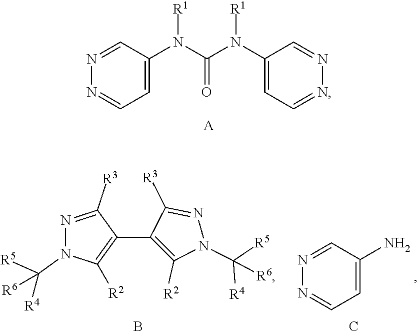

wherein R¹, R², R³, R⁴, R⁵, and R⁶, each independently is as defined hereinabove.

By-product A is a dimer which may be obtained during the process disclosed above for the preparation of the compound of formula II. Specifically, such a dimer is obtained by a side reaction wherein the starting material in the process for the preparation of the compound of formula II, i.e., the compound of formula XI, reacts with the product of said process, i.e., the compound of formula II, rather than with the carbonyl-containing group of the formula —C(O)X¹, as defined hereinabove.

Similarly, by-product B is a dimer which may be obtained by a side reaction during the process for the preparation of the compound of formula III. Specifically, such a dimer may be obtained by the Wurtz reaction, e.g., as described in Anteunis et al., Bull. Soc. Chim. Belg., 1963, 72, 787-796.

Pyridazin-4-amine, also referred to herein as by-product C, may be obtained by a side reaction wherein 4-isocyanatopyridazine reacts with water rather than with the compound of formula III, to obtain an unstable carbamic acid pyridazine derivative, i.e., pyridazin-4-ylcarbamic acid, which is then quickly loses carbon dioxide to obtain the pyridazine-based amine C. The obtained by-product C may further react with 4-isocyanatopyridazine to obtain the by-product A wherein R¹ is H.

In certain embodiments, the yield of the compound of formula I obtained by the process of the present invention is from about 5% to about 20%, from about 20% to about 40%, from about 40% to about 60%, from about 60% to about 70%, from about 70% to about 80%, from about 80% to about 90%, higher than 90%, or higher than 95%. Thus, in certain embodiments, the overall amount of the by-products A, B and/or C, when obtained in said process, constitutes up to about 30%, 25%, 20%, 15%, 10%, or 5%, by weight of the final product, or less than 5% by weight of the product.

According to the present invention, the compound of the formula I obtained by the process disclosed herein, according to any one of the embodiments above, may be in the form of a salt. Such a salt may be obtained by reacting said compound in the free base thereof, with an inorganic- or organic acid. Non-limiting examples of inorganic acids include HF, HCl, HBr, and H₂SO₄; and non-limiting examples of organic acids include a carboxylic acid such as formic acid, acetic acid, propionic acid, oxalic acid, mandelic acid, citric acid, trifluoroacetic acid, trichloroacetic acid, tartaric acid (including a chirally pure tartaric acid such as D-tartaric acid and L-tartaric acid) or a derivative thereof such as dibenzoyl tartaric acid, N-acetyl leucine, and benzoic acid, and a sulfonic acid such as toluene sulphonic acid, benzene sulphonic acid, naphthalene sulfonic acid, methansulfonic acid, and camphor sulphonic acid optionally substituted with one or more Br atoms such as 3-bromo-10-camphor sulfonic acid and 3-bromo-8-camphorsulfonic acid.

The compound of the formula I obtained by the process disclosed herein, according to any one of the embodiments above, may also be in the form of an N-oxide derivative. Such an N-oxide form may be obtained by oxidizing at least one of the nitrogen atoms of said compound. In particular such embodiments, one or both of the nitrogen atoms of the pyridazine moiety of the compound of formula I are in the form of N-oxide; and/or one or both of the nitrogen atoms of the pyrazole moiety of said compound are in the form of N-oxide, preferably one of said nitrogen atoms, more preferably the nitrogen atom adjacent to the carbon substituted by R³ group. In more particular such embodiments, the nitrogen at the para position to the amide group in the compound of the formula I is in the form of N-oxide; the nitrogen at the meta position to said amide group is in the form of N-oxide; and/or the nitrogen adjacent to the carbon substituted by R³ group.

In another aspect, the present invention provides a compound of formula III

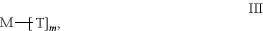

or a salt, N-oxide, tautomer, or enantiomer thereof,
wherein:
T each independently is a group of the formula:

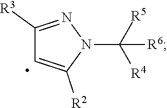

R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl;
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH;
M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminium ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex wherein M represents said metal ion connected to an additional metal ion (e.g., an alkali metal ion or alkali earth metal ion) optionally via one or more halogen anions or one or more of said T group(s);
n is an integer of 0-3;
m is an integer of 1-3; and
Cat⁺ is a cation such as an alkali metal cation,
provided that when R² is methyl, R³ is H, R⁴ is —CH(CH₃)₂, R^{S} is methyl, and R⁶ is H, M is not —B(OH)₂; 5,5-dimethyl-1,3,2-dioxaborinan-2-yl; or 4,4,5,5-tetramethyl-1,3,2-dioxaborolane-2-yl.

In certain embodiments, disclosed herein is a compound of the formula III as defined above, wherein: (i) R² is methyl, R³ is H, R⁴ is —CH(CH₃)₂, R⁵ is methyl, and R⁶ is H; (ii) R² is methyl, R³ is H, R⁴ is —C(H)(F)CH₃, R⁵ is methyl, and R⁶ is H; (iii) R² is methyl, R³ is H, R⁴ is 1-cyanocyclopropyl, R⁵ is methyl, and R⁶ is H; (iv) R² is methyl, R³ is H, R⁴ is methyl, R⁵ is methyl, and R⁶ is H; (v) R² is methyl, R³ is H, R⁴ is —CF₃, R⁵ is methyl, and R⁶ is H; (vi) R² is methyl, R³ is H, R⁴ is 1-carbamoylcyclopropyl, R⁵ is methyl, and R⁶ is H; or (vii) R² is methyl, R³ is H, R⁴ and R⁵ together represent 3,3-difluoro-1,5-pentylene, and R⁶ is H.

In particular such embodiments, m is 1, i.e., i.e., only one T group is linked to group M. In certain more particular such embodiments, M is Li ion, Cu ion, —Mg(Hal)₁, —Cu(Hal)₁, —Zn(Hal)₁, —BF₃⁻K⁺, —B(OH)₂, —B(—OiPr)₃Cat⁺, 5,5-dimethyl-1,3,2-dioxaborinan-2-yl, 4,4,5,5-tetramethyl-1,3,2-dioxaborolane-2-yl, benzo[d][1,3,2]dioxaborole-2-yl, 9-borabicyclo[3.3.1]non-9-yl, or 6-methyl-1,3,6,2-dioxazaborocane-4,8-dione-2-yl, and Hal is a halogen, preferably Cl or Br. In other more particular embodiments, M has the formula —Mg(Hal)₂Li, —Zn(Hal)₂Li, —Cu(Hal)₂Mg, or —Cu(Hal)₂Li, and together with the T group form an ate complex; Hal is a halogen, preferably Cl or Br; and the Mg, Zn or Cu ion is bound to the T group and connected to the Li or Mg ion via the halogen anions.

In other particular such embodiments, m is 2, i.e., two identical T groups are linked to group M. In more particular such embodiments, M is Mg ion or Zn ion; or M has the formula CuLi, and together with the T groups form an ate complex wherein the Cu ion is bound to each one of the T groups.

In yet other particular such embodiments, m is 3, i.e., three identical T groups are linked to group M. In more particular such embodiments, M is boron ion, Al ion, or Fe ion; or M has the formula MgLi, and together with the T groups form an ate complex wherein the Mg ion is bound to each one of the T groups.

In certain embodiments, the compound of the formula III, according to any one of the embodiments above, is in the form of a salt. In particular such embodiments, M is a boron-containing group, and such a salt may be obtained by reacting said compound in the free base thereof with an inorganic- or organic acid as defined herein.

In other embodiments, the compound of the formula III, according to any one of the embodiments above, is in the form of an N-oxide derivative. Such N-oxide derivative may be obtained by oxidizing at least one of the nitrogen atoms of said compound. In particular such embodiments, one or both of the nitrogen atoms of the pyrazole moiety of the compound of formula III are in the form of N-oxide, preferably one of said nitrogen atoms, more preferably the nitrogen atom adjacent to the carbon substituted by R³ group.

In yet another aspect, the present invention provides a compound of formula II

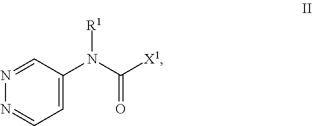

or a salt, N-oxide, or tautomer thereof,
wherein:
R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl; and
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl), provided that when R¹ is H, X¹ is not —O-phenyl.

Particular compounds of the formula II disclosed herein are those wherein R¹ is ethyl; and X¹ is halogen, —O₃SCH₃ (—O-mesyl), —O₃SC₆H₄CH₃ (—O-tosyl), —O-phenyl, and —O₃SCF₃ (—O-triflyl), or imidazole, preferably halogen, more preferably Cl.

In certain embodiments, the compound of the formula II, according to any one of the embodiments above, is in the form of a salt. Such a salt may be obtained by reacting said compound in the free base thereof with an inorganic- or organic acid as defined herein.

In other embodiments, the compound of the formula II, according to any one of the embodiments above, is in the form of an N-oxide derivative. Such an N-oxide derivative may be obtained by oxidizing at least one of the nitrogen atoms of said compound. In particular such embodiments, one or both of the nitrogen atoms of the pyridazine moiety are in the form of N-oxide. In more particular such embodiments, the nitrogen atom at the para position to the amide group in the compound of the formula II is in the form of N-oxide and/or the nitrogen at the meta position to said amide group is in the form of N-oxide.

In still another aspect, the present invention provides a compound of formula IV

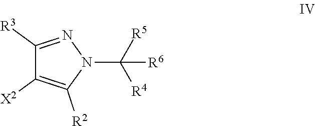

or a salt, N-oxide, or enantiomer thereof, wherein X² is halogen such as Cl and Br; and (i) R² is methyl, R³ is H, R⁴ is —CH(CH₃)₂, R⁵ is methyl, R⁶ is H; (ii) R² is methyl, R³ is H, R⁴ is —C(H)(F)CH₃, R⁵ is methyl, R⁶ is H; (iii) R² is methyl, R³ is H, R⁴ is 1-cyanocyclopropyl, R⁵ is methyl, R⁶ is H; (iv) R² is methyl, R³ is H, R⁴ is methyl, R⁵ is methyl, R⁶ is H; (v) R² is methyl, R³ is H, R⁴ is —CF₃, R⁵ is methyl, R⁶ is H; (vi) R² is methyl, R³ is H, R⁴ is 1-carbamoylcyclopropyl, R⁵ is methyl, R⁶ is H; or (vii) R² is methyl, R³ is H, R⁴ and R⁵ together represent 3,3-difluoro-1,5-pentylene, R⁶ is H.

In a further aspect, the present invention provides a composition comprising a compound of formula I:

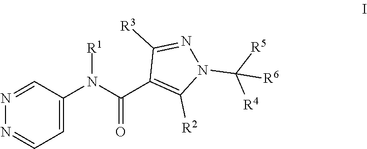

or a salt, N-oxide, tautomer, or enantiomer thereof, obtained by the process of the present invention according to any one of the embodiments above,
and at least one of the additional compounds:

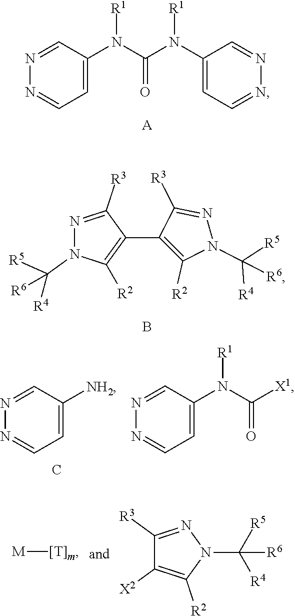

wherein:
T each independently is a group of the formula:

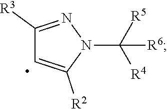

R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl;
R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl;
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH;
M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminium ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex wherein M represents said metal ion connected to an additional metal ion (e.g., an alkali metal ion or alkali earth metal ion) optionally via one or more halogen anions or one or more of said T group(s);
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl);
X² is halogen such as Cl and Br;
n is an integer of 0-3;
m is an integer of 1-3; and
Cat⁺ is a cation such as an alkali metal cation.

As explained above, the composition of the present invention may comprise the compound of the formula I in various percentages, e.g., from about 5% to about 20%, from about 20% to about 40%, from about 40% to about 60%, from about 60% to about 70%, from about 70% to about 80%, from about 80% to about 90%, or from about 90% to about 95% or more, by weight. Thus, in certain embodiments, the overall amount of said at least one additional compound comprised within said composition of the invention constitutes up to about 30%, 25%, 20%, 15%, 10%, or 5%, by weight of the overall amount of the compound of the formula I and said at least one additional compound.

Unless otherwise indicated, all numbers expressing, e.g., amounts of components or ratios between components, used in this specification, are to be understood as being modified in all instances by the term “about”. Accordingly, unless indicated to the contrary, the numerical parameters set forth in this specification are approximations that may vary by up to plus or minus 10% depending upon the desired properties to be obtained by the present invention.

The invention will now be illustrated by the following non-limiting Examples.

### EXAMPLES

### Materials and Methods

Materials. All reagents and starting materials were purchased from Thermo Fisher Scientific, Finar Limited, Avra, Merck, Symax, BLD pharma, RCP, Hindustan Platinum, CS reagent, Hychem, and Combi-Blocks.

### Example 1. Synthesis of 5-methyl-1-(3-methylbutan-2-yl)-1H-pyrazole

To a stirred solution of 3-methyl-2-butanone (50 g, 0.582 mole, HPLC purity ˜93%), was added hydrazine hydrate (31.97 g, 0.640 mole) followed by the addition of 300 mL 50% HCl at 25-30° C. The reaction mass was cooled at 0-5° C. and stirred for 1 h.

Sodium borohydride (22.8, 0.582 mole) was added to the reaction mixture at 0-5° C. Then, the reaction mass was allowed to warm to 25-30° C. and stirred for 12 h.

In another round bottom flask, solution of 4,4-dimethoxy-2-butanone (100 g, 0.756 mole) and 630 mL of 50% aqueous HCl was stirred for 2 h at 25-30° C. This solution was added to the above reaction mass (made from mixing of 3-methyl-2-butanone, hydrazine hydrate and sodium borohydride) over the period of 30 min at 0-5° C. Then, it was allowed to warm to 25-30° C. and stirred for 14 h. The reaction progress was monitored by LCMS. Then, the reaction mass was neutralized to pH 7 using saturated solution of K₂CO₃. The reaction mass was extracted with methyl tert-butyl ether (MTBE) (4×250 mL) and the combined organic layers were washed with brine (100 mL). The obtained organic layer was evaporated under vacuum to furnish crude product. The crude product was purified by column chromatography over silica gel using ethyl acetate (EtOAc)/hexane as eluent to obtain 5-methyl-1-(3-methylbutan-2-yl)-1H-pyrazole (28.9 g) as yellowish solid which approximates to about 32% yield, having a purity of about 96% as per LC-MS analysis.

¹H NMR (DMSO-d₆): δ 7.30 (d, J=8 Hz, 1H), 5.94 (d, J=8 Hz, 1H), 3.86-3.92 (m, 1H), 2.23 (s, 3H), 1.99-2.07 (m, 1H), 1.33-1.36 (m, 3H), 0.938-0.955 (d, J=6.8 Hz, 3H), 0.577-0.594 (d, J=6.8 Hz, 3H). LCMS (EI): m/z 153.2 (M+1).

### Example 2. Synthesis of 4-bromo-5-methyl-1-(3-methylbutan-2-yl)-1H-pyrazole

To the stirred solution of 5-methyl-1-(3-methylbutan-2-yl)-1H-pyrazole (20 g, 0.131 mole) in acetic acid (300 mL), bromine (6.78 mL, 0.131 mole) was added dropwise at 18-20° C. over a span of 2 h. The reaction mixture was allowed to stir at 25-30° C. for 12 h. The reaction progress was monitored by TLC. The reaction mass was quenched with saturated sodium metabisulphite solution and was kept stirring at room temperature for 1 h. Then, saturated NaHCO₃ solution was added to the reaction mass to neutralize the pH. The reaction mass was extracted with MTBE (4×100 mL) and the combined organic layers were washed with brine (100 mL). Then, the MTBE was distilled to dryness to furnish 4-bromo-5-methyl-1-(3-methylbutan-2-yl)-1H-pyrazole (25 g) as brown oil which approximates to about 82% yield, having a purity of about 90% as per LC-MS analysis.

¹H NMR (DMSO-d₆): δ 7.48 (s, 1H), 3.98-4.02 (m, 1H), 2.23 (s, 3H), 1.98-2.04 (m, 1H), 1.33-1.35 (m, 3H), 0.92-0.94 (m, 3H), 0.60-0.61 (m, 3H). LCMS (EI): m/z 231.1 (M+1).

### Example 3. Synthesis of ethyl ethyl(pyridazin-4-yl)carbamate

In a 250 mL round bottom flask, N-ethylpyridazin-4-amine (2 g, 0.016 mole) was added in dichloromethane (DCM; 50 mL), charged with triethyl amine (4.2 mL, 0.032 mole). Ethyl chlorofromate (2.27 mL, 0.024 mole) was added slowly to this reaction mass at 0-5° C., and the resulting mixture was allowed to stir at 0-5° C. for 3 h. The reaction mass was filtered off and the filtrate was evaporated under vacuum to afford the crude product. The crude product was purified by column chromatography over silica gel using methanol (MeOH)/DCM as eluent to furnish pure ethyl ethyl(pyridazin-4-yl)carbamate (1.69 g) as brown viscous liquid having a purity of about 99% as per LC-MS analysis.

¹H NMR (DMSO-d₆): δ 9.37-9.38 (m, 1H), 9.09-9.11 (m, 1H), 7.66-7.68 (m, 1H), 4.18-4.23 (m, 2H) 3.80-3.84 (m, 2H), 1.22-1.26 (m, 3H), 1.14-1.17 (m, 3H). LCMS (EI): m/z 196.1.

### Example 4. Synthesis of phenyl ethyl(pyridazin-4-yl)carbamate

In a 250 mL round bottom flask charged with triethyl amine (1.1 mL, 0.008 g), N-ethylpyridazin-4-amine (0.5 g, 0.004 mole) in EtOAc (20 mL) was added. Phenyl chlorofromate (0.6 mL, 0.0048 mole) was added slowly to this reaction mass at 0-5° C. The mixture was allowed to stir at 0-5° C. for 2 h. The reaction mass was filtered off and the filtrate was evaporated under vacuum to afford the crude product. The crude product was purified by column chromatography over silica gel using EtOAc/hexane as eluent to furnish pure phenyl ethyl(pyridazin-4-yl)carbamate (0.85 g) as brown viscous liquid which approximates to about 86% yield, having a purity of about 93% as per LC-MS analysis.

¹H NMR (DMSO-d₆): δ 9.50 (m, 1H), 9.19-9.21 (m, 1H), 7.81-7.83 (m, 1H), 7.42-7.46 (m, 1H), 7.26-7.30 (m, 3H), 3.94-3.99 (m, 2H), 1.23-1.28 (m, 3H). LCMS (EI): m/z 244.2.

### Example 5. Synthesis of N-ethyl-5-methyl-1-(3-methylbutan-2-yl)-N-(pyridazin-4-yl)-1H-pyrazole-4-carboxamide

Condition 1. In a two-necked oven dried round-bottomed flask, 4-bromo-5-methyl-1-(3-methylbutan-2-yl)-1H-pyrazole (0.828 g, 0.0036 mole) in anhydrous tetrahydrofuran (THF; 5 mL). The solution was actively purged with nitrogen gas. The flask was cooled to −78° C. 2.5M n-Butyl lithium (1.5 mL, 0.0036 mole) was added dropwise for a period of 20 min and the reaction mass was stirred at −78° C. for 30 min (Reaction Mass A).

In another oven dried two-necked round-bottomed flask, trimethylamine (0.5 mL, 0.0036 mole) was added to N-ethylpyridazin-4-amine (0.3 g, 0.0024 mole) in THF (5 mL) under nitrogen atmosphere at 25-30° C. Then, a solution of triphosgene (0.355 g, 0.0012 mole) in THF was added to the reaction mass. The resultant reaction mass was stirred at 25-30° C. for 1 h and then cooled to −78° C. (Reaction Mass B).

Reaction Mass A was taken out in 10 ml syringe and added dropwise to the stirred solution of Reaction Mass B at −78° C. The reaction mass was allowed to warm to 25-30° C. for 1 h. The crude mass was quenched with saturated solution of ammonium chloride (20 mL). The reaction mass was extracted with EtOAc (2×30 mL), washed with water (2×20 mL) and brine (20 mL). The combined organic layers were evaporated to obtain the crude product and analyzed by LCMS. The crude showed ˜47% N-ethyl-5-methyl-1-(3-methylbutan-2-yl)-N-(pyridazin-4-yl)-1H-pyrazole-4-carboxamide according to LCMS. Sample from the crude was purified by preparative HPLC (0.1% formic acid in water: acetonitrile, Column: XBridge Prep C₁₈ (250×19) mm, 5.0μ). The fractions obtained were subjected for lyophilization to get solid compound (70 mg) having a purity of about 99% as per LC-MS analysis.

¹H NMR (CDCl₃): δ 9.04-9.05 (m, 1H), 8.95-8.96 (m, 1H), 7.19-7.22 (m, 1H), 6.98 (s, 1H), 3.99-4.04 (m, 2H), 3.79-3.83 (m, 1H), 2.4 (s, 3H), 2.07-2.12 (m, 1H), 1.41 (d, J=8 Hz, 3H), 1.27 (t, J=8 Hz, 3H), 0.98 (d, J=8 Hz, 3H), 0.62 (d, J=8 Hz, 3H). 13C NMR (CDCl3): δ 164.3, 150.1, 148.3, 142.1, 140.7, 137.9, 119.6, 112.1, 59.3, 43.1, 33.0, 18.6, 18.3, 17.3, 12.5, 9.62. LCMS (EI): m/z 302.38.

Condition 2. In a two-necked oven dried round-bottomed flask, 4-bromo-5-methyl-1-(3-methylbutan-2-yl)-1H-pyrazole (0.935 g, 0.004 mole) and anhydrous THF (5 mL) was added under nitrogen atmosphere. The solution was actively purged with nitrogen gas. The flask was cooled to −78° C. with combination of dry ice/acetone. 2.5M n-Butyl lithium (2 mL, 0.005 mole) was added dropwise for a period of 20 min and stirred the reaction mass at −78° C. for 30 min (Reaction Mass A).

In another oven dried two-necked round-bottomed flask, N-ethylpyridazin-4-amine (0.25 g, 0.002 mole), TEA (0.4 mL, 0.003 mole) and DCM (3 mL) were stirred under nitrogen atmosphere. Triphosgene (0.296 g, 0.001 mole) in DCM (2 mL) was added dropwise to this reaction mixture at 25-30° C. and the reaction mixture was stirred at 25-30° C. for 2 h. The DCM was evaporated to dryness under reduced pressure below 35° C., followed by addition of dry THF (5 mL) and then, the reaction mass was cooled to −78° C. (Reaction Mass B).

Then, Reaction Mass A was taken out to a 10 ml syringe and added dropwise to the stirred solution of Reaction Mass B at −78° C. over a period of 30 min. The whole reaction mass was stirred at 25-30° C. for 12 h. The crude mass was quenched with saturated solution of ammonium chloride (20 mL). The reaction mass was extracted with EtOAc (2×30 mL), washed with water (2×20 mL) and brine (20 mL). The combined organic layer was evaporated to obtain the crude product and analyzed by LCMS. The crude showed ˜18% N-ethyl-5-methyl-1-(3-methylbutan-2-yl)-N-(pyridazin-4-yl)-1H-pyrazole-4-carboxamide according to LCMS.

Condition 3. In a two-necked oven dried round-bottomed flask, 4-bromo-5-methyl-1-(3-methylbutan-2-yl)-1H-pyrazole (0.736 g, 0.0032 mole) in anhydrous THF (5 mL) was stirred under nitrogen atmosphere and the solution was actively purged with nitrogen gas. The flask was cooled to −78° C. and 1.6 mL of 2.5M n-Butyl lithium (1.6 mL, 0.004 mole) was added dropwise to the reaction mass over 30 min at −78° C. (Reaction Mass A).

In another oven dried two-necked round-bottomed flask, N-ethylpyridazin-4-amine (0.2 g, 0.0016 mole), TEA (0.33 mL, 0.0024 mole) and DCM (3 mL) were added under nitrogen atmosphere. Triphosgene (0.236 g, 0.0008 mole) in DCM (2 mL) was added dropwise to this reaction mixture. The reaction mass was stirred at 25-30° C. for 2 h, and then cooled to −10° C. (Reaction Mass B).

Then, Reaction Mass A was taken out to 10 ml syringe and was added dropwise to the stirred solution of Reaction Mass B at −10° C. over a period of 30 min. The reaction mass was stirred at 25-30° C. for 12 h. The crude mass was quenched with saturated solution of ammonium chloride (10 mL). The reaction mass was extracted with EtOAc (2×30 mL), washed with water (2×20 mL) and brine (20 mL). The combined organic layers were evaporated to obtain the crude product and analyzed by LCMS. The crude showed ˜5% N-ethyl-5-methyl-1-(3-methylbutan-2-yl)-N-(pyridazin-4-yl)-1H-pyrazole-4-carboxamide according to LCMS.

Condition 4. Into a two-necked oven dried round-bottomed flask, 4-bromo-5-methyl-1-(3-methylbutan-2-yl)-1H-pyrazole (0.736 g, 0.0032 mole) in anhydrous THF (5 mL) was added under nitrogen atmosphere. The solution was actively purged with nitrogen gas. The flask was cooled to −78° C. 2.5M n-Butyl lithium (1.6 mL, 0.004 mole) was added dropwise to the reaction mass at −78° C. for 30 min (Reaction Mass A).

In another oven dried two-necked round-bottomed flask, N-ethylpyridazin-4-amine (0.2 g, 0.0016 mole), TEA (0.33 mL, 0.0024 mole) and DCM (3 mL) were added under nitrogen atmosphere. Triphosgene (0.236 g, 0.0008 mole) dissolved in DCM (2 mL) was added dropwise to this reaction mixture. The reaction mass was stirred at 25-30° C. for 2 h. The DCM was distilled under reduced pressure below 35° C. followed by the addition dry THF (5 mL) and the catalyst NiCl₂(PPh₃)₂ (10 mg) and then cooled to −10° C. (Reaction Mass B).

Then, Reaction Mass A was taken out to 10 ml syringe and added dropwise to the stirred solution of Reaction Mass B at −10° C. over a period of 30 min. The whole reaction mass was stirred at 25-30° C. for 12 h. The crude mass was quenched with saturated solution of ammonium chloride (10 mL). The reaction mass was extracted with EtOAc (2×30 mL), washed with water (2×20 mL) and brine (20 mL). The combined organic layers were evaporated to obtain the crude product and analyzed by LCMS. The crude showed ˜2% N-ethyl-5-methyl-1-(3-methylbutan-2-yl)-N-(pyridazin-4-yl)-1H-pyrazole-4-carboxamide according to LCMS.

Condition 5. Into a two-necked oven dried round-bottomed flask, 4-bromo-5-methyl-1-(3-methylbutan-2-yl)-1H-pyrazole (0.736 g, 0.0032 mole) in anhydrous THF (5 mL) was added under nitrogen atmosphere. The solution was actively purged with nitrogen gas. The flask was cooled to −78° C. 2.5M n-Butyl lithium (1.6 mL, 0.004 mole) was added dropwise over a period of 20 min and the reaction mass was stirred at −78° C. for 30 min (Reaction Mass A).

In another oven dried two-necked round-bottomed flask, N-ethylpyridazin-4-amine (0.2 g, 0.0016 mole), TEA (0.33 mL, 0.0024 mole) and DCM (3 mL) were added under nitrogen atmosphere. Triphosgene (0.236 g, 0.0008 mole) dissolved in DCM (2 mL) was added dropwise to this reaction mixture. The reaction mass was stirred at 25-30° C. for 2 h. The DCM was distilled under reduced pressure below 35° C. followed by the addition dry THF (5 mL) and PPh₃ (0.461 g, 0.00176 mole) and then cooled to −10° C. (Reaction Mass B).

Then, Reaction Mass A was taken out in 10 ml syringe and added dropwise to the stirred solution of Reaction Mass B at −10° C. over a period of 30 min. The whole reaction mass was stirred at 25-30° C. for 12 h. The crude mass was quenched with saturated solution of ammonium chloride (10 mL). The reaction mass was extracted with EtOAc (2×30 mL), washed with water (2×20 mL) and brine (20 mL). The combined organic layers were evaporated to obtain the crude product and analyzed by LCMS The crude showed ˜3% N-ethyl-5-methyl-1-(3-methylbutan-2-yl)-N-(pyridazin-4-yl)-1H-pyrazole-4-carboxamide according to LCMS.

Condition 6. In a two-necked oven dried round-bottomed flask, 4-bromo-5-methyl-1-(3-methylbutan-2-yl)-1H-pyrazole (1.1 g, 0.0048 mole) in anhydrous THF (5 mL) was added under nitrogen atmosphere. The solution was actively purged with nitrogen gas. The flask was cooled to −78° C. 2.5M n-Butyl lithium (2.1 mL, 0.0053 mole) was added dropwise over a period of 20 min and the reaction mass was stirred at −78° C. for 30 min (Reaction Mass A).

In another oven dried two-necked round-bottomed flask, N-ethylpyridazin-4-amine (0.3 g, 0.0024 mole), triphosgene (0.355 g, 0.0012 mole) and dry THF (5 mL) were added under nitrogen atmosphere and cooled to −78° C. 2.5M n-Butyl lithium (0.96 mL, 0.0024 mole) was added dropwise and the reaction mass was stirred at −78° C. for 1 h (Reaction Mass B).

Then, Reaction Mass A was taken in 10 ml syringe and added dropwise to the stirred solution of Reaction Mass B at −78° C. over a period of 30 min and stirred at −25° C. for 1 h. The crude mass was quenched with saturated solution of ammonium chloride (10 mL). The reaction mass was extracted with EtOAc (2×30 mL), washed with water (2×20 mL) and brine (20 mL). The combined organic layers were evaporated to obtain the crude product and analyzed by LCMS and showed ˜30% N-ethyl-5-methyl-1-(3-methylbutan-2-yl)-N-(pyridazin-4-yl)-1H-pyrazole-4-carboxamide. Sample from the crude was purified by preparative HPLC (0.1% formic acid in water: acetonitrile, Column: XBridge Prep C₁₈ (250×19) mm, 5.0μ). The fractions obtained were subjected for lyophilization to get solid compound (70 mg) having a purity of about 93% as per LC-MS analysis.

Condition 7. In a two-necked oven dried round-bottomed flask, 4-bromo-5-methyl-1-(3-methylbutan-2-yl)-1H-pyrazole (0.828 g, 0.0036 mole) in anhydrous THF (5 mL) was added under nitrogen atmosphere and the solution was actively purged with nitrogen gas. The flask was cooled to −78° C. 2.5M n-Butyl lithium (1.5 mL, 0.0036 mole) was added dropwise over a period of 20 min and the reaction mass was stirred at −78° C. for 30 min (Reaction Mass A).

In another oven dried two-necked round-bottomed flask, N-ethylpyridazin-4-amine (0.3 g, 0.0024 mole) in THF was added. Then, NaH (0.086 g, 0.0036 mole) was added to the solution. Finally, a solution of triphosgene (0.355 g, 0.0012 mole) in THF (5 mL) was added dropwise (5-10 min) under nitrogen atmosphere. The reaction mass was stirred at 25-30° C. for 1 h and then cooled to −78° C. (Reaction Mass B).

Then, Reaction Mass A was taken in 10 ml syringe and added dropwise to the stirred solution of Reaction Mass B at −78° C. over a period of 30 min. The reaction mass was stirred at 25-30° C. for 1 h. The crude mass was quenched with saturated solution of ammonium chloride (10 mL). The reaction mass was extracted with EtOAc (2×30 mL), washed with water (2×20 ML) and brine (20 mL). The combined organic layers were evaporated to obtain the crude product and analyzed by LCMS. The crude showed ˜24% N-ethyl-5-methyl-1-(3-methylbutan-2-yl)-N-(pyridazin-4-yl)-1H-pyrazole-4-carboxamide according to LCMS.

Condition 8. In a two-necked oven dried round-bottomed flask, 4-bromo-5-methyl-1-(3-methylbutan-2-yl)-1H-pyrazole (0.828 g, 0.0036 mole) in anhydrous THF (5 mL) was added under nitrogen atmosphere and the solution was actively purged with nitrogen gas. The flask was cooled to −78° C. 2.5M n-Butyl lithium (1.5 mL, 0.0036 mole) was added dropwise over a period of 20 min and the reaction mass was stirred at −78° C. for 30 min (Reaction Mass A).

In another oven dried two-necked round-bottomed flask, to a solution of N-ethylpyridazin-4-amine (0.3 g, 0.0024 mole) in DCM (5 mL), NaH (0.086 g, 0.0036 mole) was added. Then, a solution of triphosgene (0.355 g, 0.0012 mole) in DCM (2 mL) was added under nitrogen atmosphere. The reaction mass was stirred at 25-30° C. for 1 h and then cooled to −78° C. (Reaction Mass B).

Reaction Mass A was taken in 10 ml syringe and added dropwise to the stirred solution of Reaction Mass B at −78° C. over a period of 30 min. The reaction mass was stirred at room temperature for 1 h. The crude mass was quenched with saturated solution of ammonium chloride (20 mL). The reaction mass was extracted with EtOAc (2×30 mL), washed with water (2×20 mL) and brine (20 mL). The combined organic layers were evaporated to obtain the crude product and analyzed by LCMS. The crude showed ˜23% N-ethyl-5-methyl-1-(3-methylbutan-2-yl)-N-(pyridazin-4-yl)-1H-pyrazole-4-carboxamide according to LCMS.

### Example 6. Synthesis of 5-methyl-1-(3-methylbutan-2-yl)-N-(pyridazin-4-yl)-1H-pyrazole-4-carboxamide

In a two-necked oven dried round-bottomed flask, 4-bromo-5-methyl-1-(3-methylbutan-2-yl)-1H-pyrazole (0.828 g, 0.0036 mole) is added to anhydrous THF (5 mL) and stirred. The solution is actively purged with nitrogen gas. The flask is cooled to −78° C. 2.5M n-Butyl lithium (1.5 mL, 0.0036 mole) is added dropwise for a period of 20 min and the reaction mass is stirred at −78° C. for 30 min (Reaction Mass A).

In another oven dried two-necked round-bottomed flask, 4-isocyanatopyridazine (0.29 g, 0.0024 mole) is stirred in THF 5 mL under nitrogen atmosphere at 25-30° C. The resultant reaction mass is cooled to −78° C. (Reaction Mass B).

Reaction Mass A is taken out in 10 ml syringe and added dropwise to the stirred solution of Reaction Mass B at −78° C. The reaction mass is allowed to warm to 25-30° C. for 4 h. The crude mass is quenched with saturated solution of ammonium chloride (20 mL).

The reaction mass is extracted with EtOAc (2×30 mL), washed with water (2×20 mL) and brine (20 mL). The combined organic layers are evaporated to obtain the crude product and analyzed by LCMS. The crude is showing ˜50% 5-methyl-1-(3-methylbutan-2-yl)-N-(pyridazin-4-yl)-1H-pyrazole-4-carboxamide according to LCMS. Sample from the crude is purified by preparative HPLC (0.1% Formic acid in water: Acetonitrile, Column: XBridge Prep C₁₈ (250×19) mm, 5.0μ). The fractions obtained are subjected to lyophilization to get solid compound having a purity of about 95% as per LC-MS analysis.

### Example 7. Synthesis of N-ethyl-5-methyl-1-(3-methylbutan-2-yl)-N-(pyridazin-4-yl)-1H-pyrazole-4-carboxamide

In oven dried two-necked round-bottomed flask, 5-methyl-1-(3-methylbutan-2-yl)-N-(pyridazin-4-yl)-1H-pyrazole-4-carboxamide (0.65 g, 0.0024 mole) in 6 ml of THF at room temperature is dissolved. Then, NaH (0.086 g, 0.0036 mole) is added at 0° C.

Finally, a solution of ethyl iodide (0.44 g, 0.0028 mole) in THF (5 mL) is added dropwise (5-10 min) under nitrogen atmosphere. The reaction mass is allowed to warm to 25-30° C. over 12 h. The crude mass is quenched with saturated solution of ammonium chloride (20 mL). The reaction mass is extracted with EtOAc (2×30 mL), washed with water (2×20 ML) and brine (20 mL). The combined organic layers are evaporated to obtain the crude product and analyzed by LCMS. The crude is showing ˜20% N-ethyl-5-methyl-1-(3-methylbutan-2-yl)-N-(pyridazin-4-yl)-1H-pyrazole-4-carboxamide according to LCMS.

1. A process for the preparation of a compound of formula I 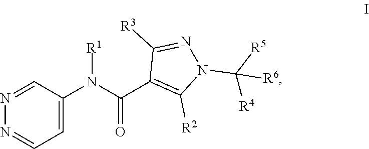
or a salt, N-oxide, tautomer, or enantiomer thereof,
wherein:
R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl;
R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl; and
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH,
said process comprising reacting a compound of formula III with either:
(i) a compound of formula II, to thereby obtain the compound of formula I; or
(ii) 4-isocyanatopyridazine, to thereby obtain the compound of formula I wherein R¹ is H, and optionally substituting said hydrogen with a group selected from (C₁-C₁₂)alkyl optionally interrupted with one or more groups each independently selected from —O—, —S—, and —N((C₁-C₁₂)alkyl)-, (C₂-C₆)alkenyl, and (C₃-C₇)cycloalkyl, 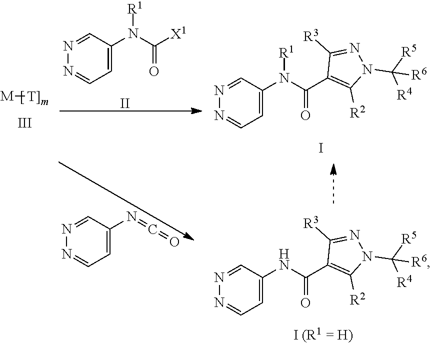
wherein:
T each independently is a group of the formula: 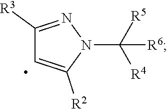
M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminium ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex, wherein M represents said metal ion connected to an additional metal ion (e.g., an alkali metal ion or alkali earth metal ion) optionally via one or more halogen anions or one or more of said T group(s);
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl);
n is an integer of 0-3;
m is an integer of 1-3; and
Cat⁺ is a cation such as an alkali metal cation.

2. The process of claim 1, carried out at a temperature of from about −100° C. to about 70° C., preferably from about −78° C. to about 25° C.

3. The process of claim 1, wherein the compound of formula III is reacted with a compound of formula II.

4. The process of claim 1, wherein the compound of formula III is reacted with 4-isocyanatopyridazine, and the compound thus obtained is reacted with a base, following by a reaction with an alkylation reagent.

5. The process of claim 4, wherein said alkylation agent is ethyl iodide, methyl iodide, ethyl methanesulfonate, trimethyloxonium tetrafluoroborate, triethyloxonium tetrafluoroborate, triethyloxonium hexachloroantimonate, bromoethane, or diethylsulfate.

6. The process of claim 1, wherein the compound of formula III is prepared from a compound of formula IV, or a mixture thereof, following the process: 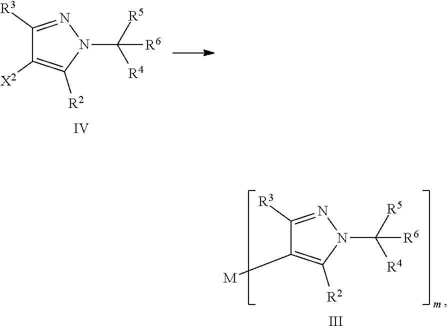
wherein:
X² is halogen such as Cl and Br; and
R², R³, R⁴, R⁵, R⁶, M, and m each independently is as defined in claim 1,
by substituting the X² group with -M-[T]ₘ₋₁.

7. The process of claim 6, wherein said reaction is carried out at a temperature of from about −100° C. to about 100° C.

8. The process of claim 6 or 7, wherein M is a group of formula —Y—Z₍ₙ₎, wherein Y is lithium ion, and n is 0, comprising reacting the compound of formula IV with n-butyl lithium or n-hexyl lithium in a solvent such as tetrahydrofuran, at a temperature of from about −78° C. to about 0° C.

9. The process of claim 1, wherein the compound of formula II is prepared from a compound of formula XI, following the process: 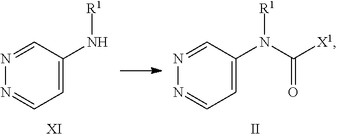
by substituting the nitrogen atom of the secondary amine group by a carbonyl-containing group of the formula —C(O)X¹, wherein R¹ and X¹ each independently is as defined in claim 1.

10. The process of claim 9, wherein said substitution is carried out by dissolving the compound of formula XI in a solvent, and then reacting said compound with a base at a temperature of from about −100° C. to about 50° C., followed by a reaction with a carbonyl-containing reagent.

11. The process of claim 10, wherein said base is trimethylamine, triethylamine, tributylamine, N,N-diisopropylethylamine, n-butyl lithium, t-butyl lithium, n-hexyl lithium, lithium diisopropylamide (LDA), potassium bis(trimethylsilyl)amide (KHMDS), sodium hydroxide (e.g., in the form of a solid or aqueous solution of at least 20% sodium hydroxide), potassium carbonate, sodium carbonate, or sodium hydride.

12. The process of claim 10, wherein said carbonyl-containing reagent is phenyl chloroformate, phosgene, 1,1,1-trichloromethylformate (diphosgene), bis(trichloromethyl) carbonate (BTC; triphosgene), or phenyl 4,5-dichloro-6-oxopyridazine-1(6H)-carboxylate.

13. The process of any one of claims 1-12, wherein:
(i) R¹ is ethyl, R² is methyl, R³ is H, R⁴ is —CH(CH₃)₂, R⁵ is methyl, and R⁶ is H;
(ii) R¹ is ethyl, R² is methyl, R³ is H, R⁴ is —C(H)(F)CH₃, R⁵ is methyl, and R⁶ is H;
(iii) R¹ is ethyl, R² is methyl, R³ is H, R⁴ is 1-cyanocyclopropyl, R⁵ is methyl, and R⁶ is H;
(iv) R¹ is ethyl, R² is methyl, R³ is H, R⁴ is methyl, R⁵ is methyl, and R⁶ is H;
(v) R¹ is ethyl, R² is methyl, R³ is H, R⁴ is —CF₃, R⁵ is methyl, and R⁶ is H;
(vi) R¹ is ethyl, R² is methyl, R³ is H, R⁴ is 1-carbamoylcyclopropyl, R⁵ is methyl, and R⁶ is H; or
(vii) R¹ is ethyl, R² is methyl, R³ is H, R⁴ and R⁵ together represent 3,3-difluoro-1,5-pentylene, and R⁶ is H.

14. The process of claim 13, wherein m is 1.

15. The process of claim 14, wherein M is Li ion, Cu ion, —Mg(Hal)₁, —Cu(Hal)₁, —Zn(Hal)₁, —BF₃⁻K⁺, —B(OH)₂, —B(—OiPr)₃Cat⁺, 5,5-dimethyl-1,3,2-dioxaborinan-2-yl, 4,4,5,5-tetramethyl-1,3,2-dioxaborolane-2-yl, benzo[d][1,3,2]dioxaborole-2-yl, 9-borabicyclo[3.3.1]non-9-yl, or 6-methyl-1,3,6,2-dioxazaborocane-4,8-dione-2-yl, and Hal is a halogen, preferably Cl or Br; or M has the formula —Mg(Hal)₂Li, —Zn(Hal)₂Li, —Cu(Hal)₂Mg, or —Cu(Hal)₂Li, and together with the T group form an ate complex, Hal is a halogen, preferably Cl or Br, and the Mg, Zn or Cu ion is bound to the T group and connected to the Li or Mg ion via the halogen anions.

16. The process of claim 13, wherein m is 2.

17. The process of claim 16, wherein M is Mg ion or Zn ion, or M together with the T groups form an ate complex wherein M has the formula CuLi, wherein the Cu ion is bound to each one of the two T groups.

18. The process of claim 13, wherein m is 3.

19. The process of claim 18, wherein M is boron ion, Al ion or Fe ion, or M together with the T groups form an ate complex wherein M has the formula MgLi, wherein the Mg ion is bound to each one of the three T groups.

20. The process of any one of claims 1-19, wherein in addition to the compound of formula I, at least one of by-products 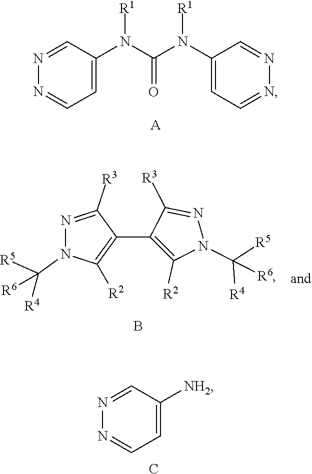
obtained,
wherein R¹, R², R³, R⁴, R⁵, and R⁶, each independently is as defined in claim 1.

21. The process of claim 20, wherein the overall amount of said at least one by-products constitutes up to about 20% by weight of the final product.

22. The process of claim 1, wherein the salt of the compound of formula I is obtained by reacting said compound in the free base form thereof with an inorganic acid such as HF, HCI, HBr, and H₂SO₄; or an organic acid such as a carboxylic acid (e.g., formic acid, acetic acid, propionic acid, oxalic acid, mandelic acid, citric acid, trifluoroacetic acid, trichloroacetic acid, tartaric acid or a derivative thereof such as dibenzoyl tartaric acid, N-acetyl leucine, and benzoic acid) and a sulfonic acid (e.g., toluene sulphonic acid, benzene sulphonic acid, naphthalene sulfonic acid, methansulfonic acid, and camphor sulphonic acid optionally substituted with one or more Br atoms such as 3-bromo-10-camphor sulfonic acid and 3-bromo-8-camphorsulfonic acid).

23. A compound of formula III 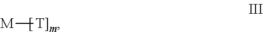
or a salt, N-oxide, tautomer, or enantiomer thereof,
wherein:
T each independently is a group of the formula: 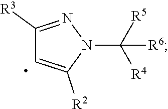
R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl;
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH;
M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminium ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex wherein M represents said metal ion connected to an additional metal ion (e.g., an alkali metal ion or alkali earth metal ion) optionally via one or more halogen anions or one or more of said T group(s);
n is an integer of 0-3;
m is an integer of 1-3; and
Cat⁺ is a cation such as an alkali metal cation,
provided that when R² is methyl, R³ is H, R⁴ is —CH(CH₃)₂, R⁵ is methyl, and R⁶ is H, M is not —B(OH)₂; 5,5-dimethyl-1,3,2-dioxaborinan-2-yl; or 4,4,5,5-tetramethyl-1,3,2-dioxaborolane-2-yl.

24. The compound of claim 23, wherein:
(i) R² is methyl, R³ is H, R⁴ is —CH(CH₃)₂, R⁵ is methyl, and R⁶ is H;
(ii) R² is methyl, R³ is H, R⁴ is —C(H)(F)CH₃, R⁵ is methyl, and R⁶ is H;
(iii) R² is methyl, R³ is H, R⁴ is 1-cyanocyclopropyl, R⁵ is methyl, and R⁶ is H;
(iv) R² is methyl, R³ is H, R⁴ is methyl, R⁵ is methyl, and R⁶ is H;
(v) R² is methyl, R³ is H, R⁴ is —CF₃, R⁵ is methyl, and R⁶ is H;
(vi) R² is methyl, R³ is H, R⁴ is 1-carbamoylcyclopropyl, R⁵ is methyl, and R⁶ is H; or
(vii) R² is methyl, R³ is H, R⁴ and R⁵ together represent 3,3-difluoro-1,5-pentylene, and R⁶ is H.

25. The compound of claim 24, wherein m is 1.

26. The compound of claim 25, wherein M is Li ion, Cu ion, —Mg(Hal)₁, —Cu(Hal)₁, —Zn(Hal)₁, —BF₃⁻K⁺, —B(OH)₂, —B(—OiPr)₃Cat⁺, 5,5-dimethyl-1,3,2-dioxaborinan-2-yl, 4,4,5,5-tetramethyl-1,3,2-dioxaborolane-2-yl, benzo[d][1,3,2]dioxaborole-2-yl, 9-borabicyclo[3.3.1]non-9-yl, or 6-methyl-1,3,6,2-dioxazaborocane-4,8-dione-2-yl, and Hal is a halogen, preferably Cl or Br; or M has the formula —Mg(Hal)₂Li, —Zn(Hal)₂Li, —Cu(Hal)₂Mg, or —Cu(Hal)₂Li, and together with the T group form an ate complex, Hal is a halogen, preferably Cl or Br, and the Mg, Zn or Cu ion is bound to the T group and connected to the Li or Mg ion via the halogen anions.

27. The compound of claim 24, wherein m is 2.

28. The compound of claim 27, wherein M is Mg ion or Zn ion; or M has the formula CuLi, and together with the T groups form an ate complex wherein the Cu ion is bound to each one of the T groups.

29. The compound of claim 24, wherein m is 3.

30. The compound of claim 29, wherein M is boron ion, Al ion or Fe ion; or M has the formula MgLi, and together with the T groups form an ate complex wherein the Mg ion is bound to each one of the T groups.

31. A compound of formula II 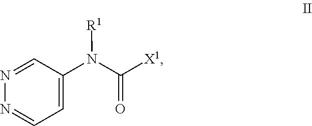
or a salt, N-oxide, or tautomer thereof,
wherein:
R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl; and
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl),
provided that when R¹ is H, X¹ is not —O-phenyl.

32. The compound of claim 31, wherein R¹ is ethyl; and X¹ is halogen, —O₃SCH₃ (—O-mesyl), —O₃SC₆H₄CH₃ (—O-tosyl), —O-phenyl, and —O₃SCF₃ (—O-triflyl), or imidazole, preferably halogen, more preferably Cl.

33. A compound of formula IV 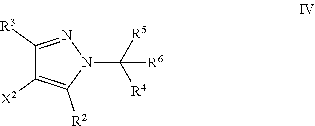
or a salt, N-oxide, or enantiomer thereof, wherein X² is halogen such as Cl and Br; and
(i) R² is methyl, R³ is H, R⁴ is —CH(CH₃)₂, R⁵ is methyl, R⁶ is H;
(ii) R² is methyl, R³ is H, R⁴ is —C(H)(F)CH₃, R⁵ is methyl, R⁶ is H;
(iii) R² is methyl, R³ is H, R⁴ is 1-cyanocyclopropyl, R⁵ is methyl, R⁶ is H;
(iv) R² is methyl, R³ is H, R⁴ is methyl, R⁵ is methyl, R⁶ is H;
(v) R² is methyl, R³ is H, R⁴ is —CF₃, R⁵ is methyl, R⁶ is H;
(vi) R² is methyl, R³ is H, R⁴ is 1-carbamoylcyclopropyl, R⁵ is methyl, R⁶ is H; or
(vii) R² is methyl, R³ is H, R⁴ and R⁵ together represent 3,3-difluoro-1,5-pentylene, R⁶ is H.

34. A composition comprising a compound of formula I: 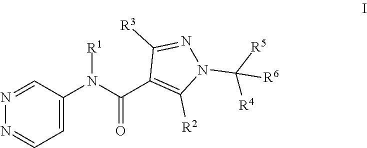
or a salt, N-oxide, tautomer, or enantiomer thereof, obtained by the process of any one of claims 1-22,
and at least one additional compound selected from: 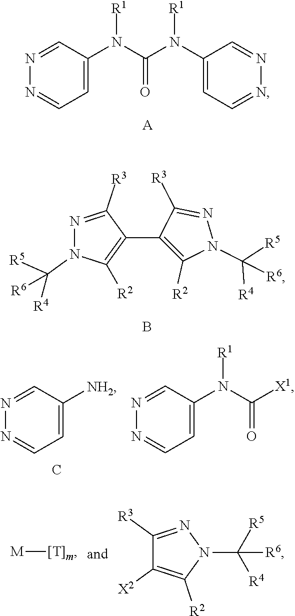
wherein:
T each independently is a group of the formula: 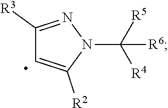
R¹ is H, (C₁-C₁₂)alkyl optionally interrupted with one or more groups each independently selected from —O—, —S—, and —N((C₁-C₁₂)alkyl)-, (C₂-C₆)alkenyl, or (C₃-C₇)cycloalkyl;
R² and R³ each independently is H, (C₁-C₁₂)alkyl optionally interrupted with one or more groups each independently selected from —O—, —S—, and —N((C₁-C₁₂)alkyl)-, or (C₂-C₆)alkenyl;
R⁴, R⁵, and R⁶ each independently is H, (C₁-C₁₂)alkyl optionally interrupted with one or more groups each independently selected from —O—, —S—, and —N((C₁-C₁₂)alkyl)-, (C₂-C₆)alkenyl, (C₁-C₆)haloalkyl, (C₃-C₇)cycloalkyl, (C₃-C₇)heterocyclyl, or any two of R⁴, R⁵, and R⁶ together with the carbon atom to which they are attached form a 5-7 membered ring optionally substituted with one or more groups each independently selected from halogen, —CN, —COOH, and —NO₂, wherein said cycloalkyl and heterocyclyl each independently is optionally substituted with one or more groups each independently selected from —CN, —C(O)NH₂, halogen, —NO₂, —COOH;
M is a group of formula —Y—Z₍ₙ₎, wherein Y is boron ion, or a metal ion having an oxidation state of at least one such as an alkali metal (e.g., lithium, sodium, potassium) ion, an alkali earth metal (e.g., magnesium) ion, aluminium ion, and a transition metal (e.g., copper, zinc, iron) ion, and Z represents a halogen anion; or
M is a boron-containing group selected from —B(halogen)₃⁻Cat⁺, —B(OR′)₃⁻Cat⁺, —B(—O—C(O)—CH₂—N(CH₃)—CH₂—COO—), —B(OH)₂, —B(OH)₃⁻Cat⁺, —BR′(OH), —B(R′)₂, —BR′(OR′), B(OH)(OR′), and —B(OR′)₂, wherein R′ each independently is (C₁-C₆)alkyl, (C₂-C₆)alkenyl, or (C₃-C₇)cycloalkyl, or the two R's together with the boron atom to which they are attached form a 5-9 membered ring optionally substituted with one or more groups each independently selected from (C₁-C₆)alkyl, (C₂-C₆)alkenyl, ═O, —O—(C₁-C₆)alkyl, or —O—(C₂-C₆)alkenyl; or
M together with the T group(s) form an ate complex wherein M represents said metal ion connected to an additional metal ion (e.g., an alkali metal ion or alkali earth metal ion) optionally via one or more halogen anions or one or more of said T group(s);
X¹ is a leaving group such as halogen, imidazole, —O₃SCH₃ (—O-mesyl), —O₃SC₆H₄CH₃ (—O-tosyl), —O-phenyl, and —O₃SCF₃ (—O-triflyl);
X² is halogen such as Cl and Br;
n is an integer of 0-3;
m is an integer of 1-3; and
Cat⁺ is a cation such as an alkali metal cation.

35. The composition of claim 34, wherein the amount of said at least one additional compound constitutes up to about 20% by weight of the overall amount of the compound of formula I and said at least one additional compound.
